(19) United States
(12) Patent Application Publication
(10) Pub. No.: US 2005/0148565 A1
(43) Pub. Date: Jul. 7, 2005

(54) Use of aminoalkyloxy derivatives of 1,3,5(10)-Estratrien and -Estratetraene steroids in the treatment of breast cancer

(76) Inventor: John S. Cooperwood, Tallahassee, FL (US)

Correspondence Address: FLORIDA A&M UNIVERSITY, 2077 E. PAUL DIRAC DRIVE, TALLAHASSEE, FL, 32310, US

(21) Appl. No.: 10/835,312

(22) Filed: Apr. 28, 2004

Related U.S. Application Data
(60) Provisional application No. 60/466,521, filed on Apr. 29, 2003.

(51) Int. Cl. A61K 31/58; A61K 31/56

(52) U.S. Cl. 514/176; 514/182

(57) ABSTRACT

A method of treating cancer in warm-blooded animals by administering a safe and effective amount of a pharmaceutical composition comprising compounds selected from the group consisting of the compounds of Structure I 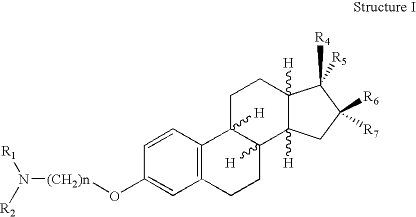 Wherein:
R₁ and R₂ are individually selected from the group consisting of alkyl groups containing 1 to 8 carbon atoms, taken together with nitrogen form a saturated 5 to 6 ring heterocycle, R₃ is α or β methyl, n is an integer from 2 to 10,
R₄ is selected from the group consisting of hydrogen, hydroxy and acyloxy of a organic carboxylic acid of up to 10 carbon atoms or taken together with the nitrogen form a saturated 5 to 6 ring heterocycle optionally containing a second nitrogen or oxygen in the ring, carboxylic acid and
R₅ selected from the group consisting of hydrogen, hydroxy, acyloxy of a organic carboxylic acid up to 10 carbons atoms and alkyl, alkenyl and alkynyl of up to 10 carbons,
R₆ and R₇ are individually selected from the group consisting of hydrogen, hydroxy and acyloxy of a organic carboxylic acid of up 10 carbons and
R₄ and R₇ are hydrogen and their non-toxic, including pharmaceutically acceptable acid addition salts.

### RELATED APPLICATIONS

This application claims the benefit of provisional patent application No. 60/466,521 filed Apr. 29, 2003.

### GOVERNMENTAL INTEREST

This invention was supported with government funding awarded by the National Institutes of Health under Grant No. RR03020-18-ARC. The government may have certain rights in the present invention.

### BACKGROUND

An embodiment of the invention is the method of using aminoalkyloxy derivatives of 1,3,5(10)-Estratrien and 1,3,5(10) 16-Estratetraene steroids as selective estrogen receptor modulators for the treatment of cancer. These compounds consist of various aminoalkyloxy derivatives of 1,3,5(10)-Estratrien and 1,3,5(10) 16-Estratetraene steroids are used as agents in the treatment of breast cancer in warm-blooded animals. Selective estrogen receptor modulators (SERMs) are a group of compounds that act as estrogen receptor agonists in some tissues and as estrogen receptor antagonists in others. SERMs interact with receptors by diffusing into the cell and binding to estrogen receptor α or β subunits. During this process, there is a conformational change to the receptors resulting in dimerization. It is this dimerization of receptors that facilitates binding to the promoter regions on DNA. The conformational change of receptors, particularly the position of helix 12, dictates whether the SERM is agonist or antagonist causing activation or suppression of estrogen target genes.

Currently, the SERMs consist of two main chemical classes: the triphenylethylene derivatives and benzothiophene derivatives. The FDA approved SERMs, used in the treatment of breast cancer, are tamoxifen and toremifene. Another SERM, raloxifene is currently approved for osteoporosis. Tamoxifen has been associated with endometrial cancer due to its estrogenic activity within the uterus while raloxifene lacks estrogenic activity within the uterus. It is apparent that there is a need to find a SERM with antiestrogenic activity and minimal side effects.

Estrogen is thought to be involved in the triggering of breast cancer as well as other cancers due to improper or excessive signaling. Tamoxifen as well as other SERMs is believed to block this triggering affect of estrogen. Over the years, tamoxifen has became the agent of choice in the treatment of all stages of breast cancer. Tamoxifen is the most effective against Estrogen receptor positive breast cancers. Clinical trials have shown that tamoxifen, when use as an adjuvant therapy, can increase the survival rate of women with estrogen receptor positive tumors and in women whose estrogen receptor status is not known. Nevertheless, tamoxifen is associated with serious side effects, such as thromboembolic events, vasomotor symptoms, and an increased risk of developing endometrial cancer and cataracts.

Toremifene is another FDA approved SERM used in the treatment of advanced breast cancer in postmenopausal women. Toremifene's efficacy is very similar to that of tamoxifen against estrogen positive metastatic breast cancer. In a worldwide phase III clincal trial comparing toremifene with tamoxifen, results indicated that the two drugs had very similar response rate with 44% for tamoxifen (20 mg/day), while the response rates for toremifene were 50% and 48% at 60 and 200 mg/day, respectively. These results indicated that both drugs have similar efficacy in treating advanced breast cancer. As with tamoxifen, toremifene has also been studied as an adjuvant therapy. In this multi-center clinical trial, toremifene was not found to exhibit a statistically significant reduction in recurrence of breast cancer. Surprisingly, toremifene has been associated with fewer side effects in comparison with tamoxifen. This advantage may be pivotal in the approval by FDA as an adjuvant therapeutic treatment for breast cancer. Raloxifene is a SERM approved by the FDA in treatment and prevention of osteoporosis. It has estrogen antagonist activity in breast tissue. Unfortunately, preclincal studies indicated that it has no advantage over tamoxifen in treating postmenopausal women with advanced breast cancer. In an addition, raloxifene is not effective against tamoxifen resistant breast cancer. But it is still being considered as a prophylactic therapy against cancer in high-risk women. Based upon the in vitro studies, the aminoalkyloxy derivatives of 1,3,5(10)-Estratrien and 1,3,5(10) 16-Estratetraene steroids are effective at inhibiting the growth of cancer cells. Particularly the growth of breast cancer cells such as MCF-7 cells and may be effective against endometrial cancer cells.

### DESCRIPTION

The method of use disclosed is directed to various class of aminoalkyloxy derivatives of 1,3,5(10)-Estratrien and 1,3,5(10) 16-Estratetraene steroids as agents in the treatment of cancer in warm-blooded animals acting the same or similarly as a selective estrogen receptor modulators upon MCF-7 cells. The relevant compounds used in the treatment of cancer are described according to the following generic structure:

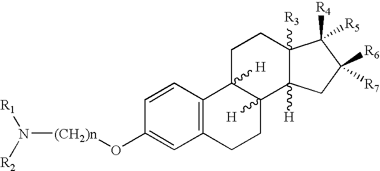

Steroids were purchased from Steroloids Inc. (Newport, R.I.). All solvents were obtain from Fisher Scientific International Inc. and were of analytical grade. Other chemicals were either obtained from Sigma-Aldrich Company or Fisher Scientific International Inc. ¹H NMR spectra were obtained with a Brucker 300 MHz spectrometer or Brucker 500 MHz spectrometers and chemical shifts are reported in parts per million (ppm) using tetramethylsilane as an internal reference.

The following procedure was used to synthesize the compounds. Sodium metal (3 mole equivalent) was dissolved in 50 mL of ethanol then steroid (1 mole equivalent) was added. To the resulting solution, the alkylamine chloride (1.2 mole equivalent) was added and reflux for 17 hours. The mixture was then filtered and the filtrate was concentrated to dryness under reduced pressure. The residue was re-dissolved in 150 mL of ethyl acetate and washed with 0.1 N aqueous hydrochloric acid (2×100 mL), then 2 N sodium hydroxide (2×100 mL) and water (100 mL). The organic layer was dried over magnesium sulfate, filtered and concentrated under reduced pressure to obtain the residue. The residue was dissolved in 100 mL of diethyl ether and acidified with gaseous hydrochloric acid to reach a pH of 3. The acidified mixture was filtered, washed with ether and solid collected. The following is a illustrative list of the compounds, but is not meant to be an exhaustive list of the compounds falling within the broad scope of this disclosure.

The HCl salts of the compounds were prepared using methanolic HCl solution. trans-4-Hydroxytamoxifen was purchased from Sigma-Aldrich company and its HCl salt was prepared using methanolic HCl solution due to avoid isomerization which occurs with the basic free amine species. The HCl salts of the compounds of structures I-III were compared with trans-4-hydroxytamoxifen in groups of four. However, preparation of the compounds is not necessarily limited to salt forms, but may include free base forms.

### EXAMPLE 1

### 3-[2-diethylamino]-ethoxy]-]-Δ^{1,3,5(10)}-estrien-17α-ethynyl-17β-ol

Using the general procedure, the product was obtained in 28% yield as a solid.

¹H-NMR (500 MHz, d₆-DMSO) δ 0.74 (s, 3H), 1.24 (m, 18H), 1.72 (m, 7H), 2.0 (m, 3H) 2.76 (br s, 2H), 6.69 (m, 2H) 7.2 (d, 1H, J=6 Hz), 7.68 (s, 1H), 10.09 (br s, 1H).

### EXAMPLE 2

### 3-[2-morpholinyl]-ethoxy]-]-Δ^{1,3,5,(10)}-estrien-17α-ethynyl-17β-ol

Using the procedure described above, the product was obtained in 64% yield as a solid.

¹H-NMR (500 MHz, d₆-DMSO) δ 0.74 (s, 3H), 1.3 (m, 4H), 1.7 (m, 7H) 2.0 (m, 2H), 2.3 (d, 1H, J =12 Hz), 2.76 (br s, 2H), 3.42 (m, 4H), 3.87 (m, 4H), 4.37 (br s, 2H), 5.34 (br s, 1H), m 6.68 (m, 2H), 7.19 (d, 2H, J=6 Hz), 11.55 (br s, 1 H)

### EXAMPLE 3

### 3-[2-dimethylamino]-ethoxy]-]-Δ^{1,3,5(10)}-estrien-17α-ethynyl-17β-ol

Using the procedure described above, the product was obtained in 25% yield as a solid.

¹H-NMR (500 MHz, d₆-DMSO) δ 0.74 (s, 3H), 1.27 (m, 10 H), 1.7 (m, 8H), 2.08 (m, 2H) 2.32 (m, 1H), 4.2 (m, 3H), 6.69 (m, 3H), 7.19 (d, 1H J=9 Hz), 7.68 (s 1H), 10.48 (br s, 1H)

### EXAMPLE 4

### 3-[2-pyrrolidinylamino]-ethoxy]-]-Δl ^{1,3,5(10 )}-estrien-17α-ethynyl-17,β-ol

Using the procedure described above, the product was obtained in 35% yield as a solid.

¹H-NMR (500 MHz, d₆-DMSO) δ 0.74 (s, 3H), 1.3 (m, 5H), 2.1-1.88 (m, 14 H)2.31 (m, 1H), 2.77 (br s, 3H), 3.07 (br s, 3H) 4.26 (br s, 2H) 5.33 (br s 1H) 6.72 (m, 2H), 7.19 (d, 1H J=9Hz), 10.54 (br s, 1H)

### EXAMPLE 5

### 3-[2-piperidinyl]-ethoxy]-]-Δ^{1,3,5(10)}-estrien-17α-ethynyl-17δ-ol

Using the procedure described above, the product was obtained in 60% yield as a solid.

¹H-NMR (500 MHz, d₆-DMSO) δ 0.74 (s, 3H), 1.34 (m, 6H), 1.7 (m, 12H), 2.08 (m, 2H), 2.31 (m, 1H), 2.76 (br s, 1H), 2.97 (m, 2H), 3.44 (m, 4H), 4.30 (br s, 1H), 5.35 (br s, 1H) 6.68 (m, 2H), 7.19 (d, 1H J=9 Hz), 9.94 (br s, 1H)

### EXAMPLE 6

### 3-[2-diisopropylamino]-ethoxy]-]-Δ^{1,3,5(10)}-estrien-17α-ethynyl-17β-ol

Using the procedure described above, the product was obtained in 43% yield as a solid

¹H-NMR (500 MHz, d₆-DMSO) δ 0.68 (s, 3H), 1.13 (t, 2H J=24 Hz), 1.28 (m, 12H), 1.6(m, 5H), 1.8 (m, 5H), 2.07 (m, 2H), 3.48 (m, 3H), 2.30 (m, 2H), 2.77 (br s, 2H), 3.22 (m, 2H) 6.68 (m, 2H), 7.20 (d, 1H J=6 Hz), 9.80 (br s, 1H)

### EXAMPLE 7

### 3-[2-diethylamino]-ethoxy]-]-Δ^{1,3,5(10)}-estrien-17-one

Using the procedure described above, the product was obtained in 53% yield as a solid

¹H-NMR (300 MHz, d₆-DMSO) θ 0.81 (s, 3H), 1.35 (m, 10 H), 2.1 (m, 8H), 2.08 (m, 3H) 2.32 (m, 1H), 2.81 (br s, 3 H) 3.4 (br s, 3H), 6.70 (m, 2H), 7.19 (d, 1H J=9 Hz), 10.70 (br s, 1H)

### EXAMPLE 8

### 3-[2-dimethylamino]-ethoxy]-]-Δ^{1,3,5(10)}-estrien-17-one

Using the procedure described above, the product was obtained in 42% yield as a solid

¹H-NMR (300 MHz, d₆-DMSO) δ 0.81 (s, 3H), 1.37 (m, 8 H), 1.7 (m, 8H), 2.2 (m, 2H) 2.79 (br s, 6H), 6.73 (m, 2H), 7.19 (d, 1H J=9 Hz), 7.68 (s 1H), 10.72 (br s, 1H)

### EXAMPLE 9

### 3-[2-pyrrolidinylamino]-ethoxy]-]-Δ^{1,3,5(10)}-estrien-17-one

Using the procedure described above, the product was obtained in 60% yield as a solid

¹H-NMR (300 MHz, d₆-DMSO) δ 0.81 (s, 3H), 1.49 (m, 7 H), 1.7 (m, 8H), 2.81 (m, 2H) 3.06 (m, 2H), 3.5 (br s, 3H) 4.2 (m, 2H), 6.69 (m, 2H), 7.19 (d, 1H J=6 Hz), 7.68 (s 1H), 11.07 (br s, 1H)

### EXAMPLE 10

### 3-[2-piperidinyl]-ethoxy]-]-Δ^{1,3,5(10)}-estrien-17-one

Using the procedure described above, the product was obtained in 69% yield as a solid

¹H-NMR (300 MHz, d₆-DMSO) δ 0.81 (s, 3H), 1.35 (m, 12 H), 2.09 (m, 2H), 2.4 (m, 2H), 2.9 (m, 5H), 3.4 (m, 4H), 4.3 (m, 3H), 6.7 (m, 2H), 7.19 (d, 1H J=9 Hz), 10.89 (br s, 1H)

### EXAMPLE 11

### 3-[2-diisopropylamino]-ethoxy]-]-Δ^{1,3,5(10)}-estrien-17-one

Using the procedure described above, the product was obtained in 44% yield as a solid

¹H-NMR (300 MHz, d₆-DMSO) δ 0.81 (s, 3H), 1.24 (m, 14H), 1.3 (m, 12H), 1.8 (m, 2H), 2.12 (m, 2H), 4.2 (m, 2H) 6.67 (m, 2H) 7.2 (d, 1H, J=9 Hz), 7.68 (s, 1H), 9.55 (br s, 1H)

### EXAMPLE 12

### 3-[2-morpholinyl]-ethoxy]-]-Δ^{1,3,5(10)}-estrien-17-one

Using the procedure described above, the product was obtained in 65% yield as a solid

¹H-NMR (300 MHz, d₆-DMSO) δ 0.81 (s, 3H), 1.3 (m, 10H), 2.81 (m, 4H), 3.4 (m, 4H), 3.8 (m, 6H), 4.24 (t, 2H J=9 Hz), 4.37 (br s, 2H), 6.69 (m, 2H) 7.2 (d, 1H, J=9 Hz), 11.39 (br s, 1H)

### EXAMPLE 13

### (17δ)-3-[2-diethylamino]-ethoxy]-]-Δ^{1,3,5(10)}-estrien-17-ol

Using the procedure described above, the product was obtained in 83% yield as a solid

1H-NMR (300 MHz, d₆-DMSO) δ 0.65 (s, 3H), 1.3(m, 10H), 1.82 (m, 3H), 2.08 (m, 2H), 2.24 (m, 2H), 2.76 (br s, 2H), 3.6 (m, 7H), 4.29 (br s, 3H), 6.63 (m, 2H) 7.2 (d, 1H, J=4 Hz), 7.68 (s, 1H), 9.90 (br s, 1H)

### EXAMPLE 14

### (17β)-3-[2-dimethylamino]-ethoxy]-Δ^{1,3,5(10)}-estrien-17-ol

Using the procedure described above, the product was obtained in 74% yield as a solid

1H-NMR (300 MHz, d₆-DMSO) δ 0.65 (s, 3H), 1.28 (m, 8H), 1.25 (m, 3H), 1.8 (m, 5H), 2.08 (m, 2H), 2.25 (m, 2H), 2.79 (br s, 6H) 3.26 (m, 4H) 6.68 (m, 2H) 7.19 (d, 1H, J=6 Hz), 7.68 (s, 1H), 10.54 (br s, 1H)

### EXAMPLE 15

### (17δ)-3-[2-pyrrolidinylamino]-ethoxy]-]-Δ^{1,3,5(10)}-estrien-17-ol

Using the procedure described above, the product was obtained in 66% yield as a solid

1H-NMR (300 MHz, d₆-DMSO) δ 0.65 (s, 3H), 1.27 (m, 7H), 1.72 (m, 8H), 3.26 (m, 2H) 3.41 (m, 6H), 4.2 (m, 4 H) 5.34 (br s, 1H), 6.7 (m, 2H) 7.2 (br s, 1H), 7.68 (s, 1H), 10.81 (br s, 1H)

### EXAMPLE 16

### (17δ)-3-[2-piperidinyl]-ethoxy]-]-Δ^{1,3,5(10)}-estrien-17-ol

Using the procedure described above, the product was obtained in 39% yield as a solid

1H-NMR (300 MHz, d₆-DMSO) δ 0.81 (s, 3H), 0.85 (br s, 3H), 1.35 (m, 10H), 1.9 (m, 8H), 2.12 (m, 2H), 2.4 (m, 2H), 4.25 (m, 4H), 5.35 (br s, 1H) 6.7 (m, 2H), 7.19 (d, 1H J=9 Hz), 7.68 (s, 1H), 10.89 (br s, 1H)

### EXAMPLE 17

### (17δ)-3-[2-diisopropylamino]-ethoxy]-]-Δ^{1,3,5(10)}-estrien-17-ol

Using the procedure described above, the product was obtained in 73% yield as a solid

1H-NMR (300 MHz, d₆-DMSO) δ 0.60 (s, 3H), 1.22 (m, 14H), 1.7-2.3 (m, 10H), 2.7 (br s, 2H), 3.15 (br s, 4H), 4.3 (m, 3H) 6.69 (m, 2H), 7.2 (d, 1H J=9 Hz), 7.68 (s, 1H), 10.38 (br s, 1H)

### EXAMPLE 18

### (17δ)-3-[2-morpholinyl]-ethoxy]-]-Δ^{1,3,5(10)}-estrien-17-ol

### Using the procedure described above, the product was obtained in 63% yield as a solid

1H-NMR (300 MHz, d₆-DMSO) δ 0.64 (s, 3H), 1.2 (m, 4H), 1.8 (m, 3H), 2.12 (m, 2H), 2.24 (m, 2H) 2.09 (m, 3H), 2.75 (br s, 3H) 3.15 (m, 3H), 3.47 (m, 5H), 3.88 (m, 4H), 6.68 (m, 2H), 7.18 (d, 1H J=9 Hz), 7.68 (s, 1H), 11.55 (br s, 1H).

The above compounds that were made and analyzed are included in the following structures. The compounds are used to treat cancer in warm-blooded animals by administering a safe and effective amount of a pharmaceutical composition comprising compounds selected for the group consisting of compounds according to Structures II-IV. 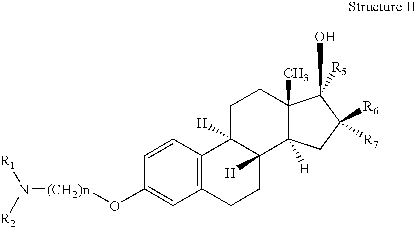
Wherein:
R₁ and R₂ are individually alkyl of 1 to 8 carbon atoms, taken together with the nitrogen form a saturated 5 to 6 ring heterocycle,
n is an integer of 2 to 10 and
R₅ is hydrogen
R₆ is hydrogen
R₇ are hydrogen
including pharmaceutically acceptable acid salts. 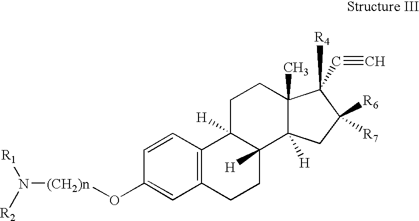
   wherein:
R₁ and R₂ are individually selected from the group consisting of alkyl groups containing 1 to 8 carbon atoms, taken together with nitrogen form a saturated 5 to 6 ring heterocycle,
n is an integer of 2 to 10 and either R₄ is ethynyl,
R₆ is hydrogen,
R₇ are hydrogen
including their pharmaceutically acceptable acid salts. 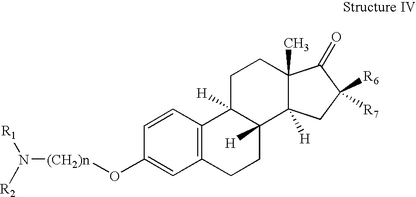
   Wherein:
R₁ and R₂ are individually selected from the group consisting of alkyl groups containing 1 to 8 carbon atoms taken together with nitrogen form a saturated 5 to 6 ring heterocycle,
n is an integer of 2 to 10,
R₄ and R₅ form a double-bond with oxygen (═O),
R₆ is hydrogen
R₇ is hydrogen

The compounds selected are those having attached groups wherein R₁ and R₂ are individually selected from the group consisting of alkyl of 1 to 8 carbon atoms or taken together with the nitrogen form a saturated 5 to 6 ring heterocycle. Optionally, if a ring is formed R₁ and R₂ may contain a second nitrogen or oxygen in the ring, R₃ is alpha or beta-methyl, n is an integer from 2 to 10, R₄ and R₅ together are double bonded or R₄ is from a group consisting of hydrogen, hydroxyl (—OH) and acyloxy of an organic carboxylic acid of up to 12 carbon atoms and R₅ is from group consisting of hydrogen, hydroxyl (—OH), acyloxy of an organic carboxylic acid of up to 12 carbon atoms and alkyl, alkenyl and alkynyl of up to 8 carbon atoms, R₆ and R₇ are double-bonded oxygen or are from group consisting of hydrogen, hydroxyl (—OH) and acyloxy of an organic carboxylic acid of up to 12 carbon atoms or R₅ and R₆ form a double bond and R₄ and R₇ are hydrogen and their non-toxic, pharmaceutically acceptable acid addition salts in an amount sufficient for the treatment of breast cancer.

Examples of R₁, R₂ and R₅ as alkyl of 1 to 8 carbon atoms consist of methyl, ethyl, propyl, isopropyl, butyl, isobutyl, tert-butyl, n-pentyl, n-hexyl, 2-methylpentyl, 2,3-dimethylbutyl, n-heptyl, 2-methylhexyl, 2,2,-dimethyl-pentyl, 3,3-dimethyl-pentyl, 3-ethylpentyl, n-octyl, 2,2-dimethylhexyl, 3,3-dimethylhexyl and 3-methyl-3-ethylpentyl. In particular interest methyl, ethyl, isopropyl.

In the case R₁ and R₂ form a heterocyclic ring with nitrogen consisting of 5 to 6 members with heteroatom chosen from oxygen and nitrogen optionally. In particularly piperidinyl, piperazinyl, morpholinyl or pyrrolindinyl ring system are encompassed in this invention.

Examples of acyloxyl of an organic carboxylic acid of up to 12 carbon atoms formyloxyl, acetyloxy, propionyloxyl, butyryloxyl, hexanoyloxyl and benzoyloxyl

Examples of alkenyl having at most 8 carbons are vinyl, allyl, 1-propenyl, butenyl, pentenyl or hexenyl

Examples of alkynyl having at most 8 carbon atoms are ethynyl, propargyl, butynyl, pentynyl or hexynyl.

Examples of suitable acids for the non-toxic, pharmaceutically acceptable acid salts are hydrochloric acid, hydrobromic acid, nitric acid, sulfuric acid, phosphoric acid, acetic acid, formic acid, propionic acid, benzoic acid, maleic acid, furrmaric acid, succinic acid, tartaric acid, citric acid, oxalic acid, glyoxylic acid, aspartic acid, alkane sulfonic acids such as methane or ethane sulfonic acid, arylsulfonic acids such as benzene acid or p-toluene sulfonic acids such as p-toluene sulfonic or benzene acid and arylcarboxylic acids. In particular hydrochloric

The following structures depict suitable compounds for treating cancer in warm-blooded animals by administering a safe and effective amount of one or more compounds are selected for the group consisting of compounds according to Structure II. 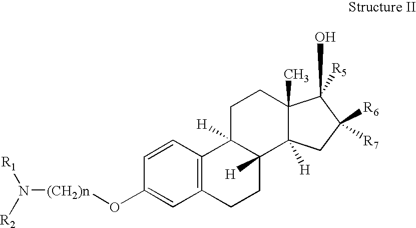
Wherein:
R₁ and R₂ are individually alkyl of 1 to 8 carbon atoms, taken together with nitrogen form a saturated 5 to 6 ring heterocycle,
n is an integer of 2 to 10 and
R₅ is hydrogen
R₆ is hydrogen
R₇ are hydrogen
including pharmaceutically acceptable acid salts. The following compounds included in the scope of this invention include, but are not limited to:
17δ)-3-[2-dimethylamino]-ethoxy]Δ^{1,3,5(10)}-estrien-17-ol
(17δ)-3-[2-diethylamino]-ethoxy]-]-Δ^{1,3,5(10)}-estrien-17-ol
(17δ)-3-[2-diisopropylamino]-ethoxy]-]-Δ^{1,3,5(10)}-estrien-17-ol
(17δ)-3-[2-morpholinyl]-ethoxy]-]-Δ^{1,3,5(10)}-estrien-17-ol
(17δ)-3-[2-piperidinyl]-ethoxy]-]-Δ^{1,3,5(10)}-estrien-17-ol
(17δ)-3-[2-pyrrolidinylamino]-ethoxy]-]-Δ^{1,3,5(10)}-estrien-17-ol 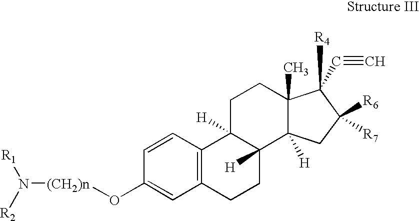
   wherein:
R₁ and R₂ are individually selected from the group consisting of alkyl groups containing 1 to 8 carbon atoms, taken together with the nitrogen form a saturated 5 to 6 ring heterocycle,
n is an integer of 2 to 10 and either R₄ is ethynyl,
R₆ is hydrogen,
R₇ are hydrogen
including their pharmaceutically acceptable acid salts. The following compounds included in the scope of this invention include, but are not limited to:
3-[2-dimethylamino]-ethoxy]-]-Δ^{1,3,5(10)}-estrien-17α-ethynyl-17β-ol
3-[2-diethylamino]-ethoxy]-]-Δ^{1,3,5(10)}-estrien-17α-ethynyl-17β-ol
3-[2-diisopropylamino]-ethoxy]-]-Δ^{1,3,5(10)}-estrien-17α-ethynyl-17β-ol
3-[2-morpholinyl]-ethoxy]-]-Δ^{1,3,5(10)}-estrien-17α-ethynyl-17β-ol
3-[2-piperidinyl]-ethoxy]-]-Δ^{1,3,5(10)}-estrien-17α-ethynyl-17β-ol
3-[2-pyrrolidinylamino]-ethoxy]-]Δ^{1,3,5(10)}-estrien-17α-ethynyl-17β-ol 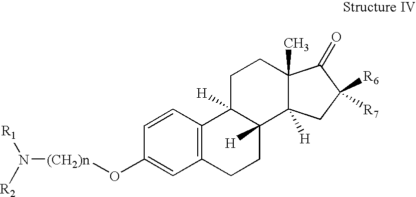
   Wherein:
R₁ and R₂ are individually selected from the group consisting of alkyl groups containing 1 to 8 carbon atoms taken together with nitrogen form a saturated 5 to 6 ring heterocycle,
n is an integer of 2 to 10,
R₄ and R₅ form a double-bond with oxygen (═O),
R₆ is hydrogen
R₇ is hydrogen
   The following compounds included in the scope of this invention include, but are not limited to:
3-[2-dimethylamino]-ethoxy]-]Δ^{1,3,5(10)}-estrien-17-one
3-[2-diethylamino]-ethoxy]-]Δ^{1,3,5(10)}-estrien-17-one
3-[2-diisopropylamino]-ethoxy]-]-Δ^{1,3,5(10)}-estrien-17-one
3-[2-morpholinyl]-ethoxy]-]-Δ^{1,3,5(10)}-estrien-17-one
3-[2-piperidinyl]-ethoxy]-]-Δ^{1,3,5(10)}-estrien-17-one
3-[2-pyrrolidinylamino]-ethoxy]-]-Δ^{1,3,5(10)}-estrien-17-one

The wavy lines in positions 8, 9 and 14 indicate that the hydrogens are in position (8β, 9α, 14α) or 8α, 9β, 14β).

The wavy lines in position 17 indicate that the R₄ and R₅ groups are in position α or β . R₄ is in alpha position and R₅ is in β position or R₄ is β position or R₅ is in α position.

Among the preferred compounds of this invention are those wherein R.sub.3 is a .beta.-methyl, those of the formula wherein R₁, R₂, R₅ and n are defined as above, those of the formula wherein R₁ and R₂ are individually alkyl of 1 to 8 carbons atoms, n is defined as above and either R₄ is hydrogen and R₅ is hydroxy (—OH) or R₄ and R₅ are both hydrogens or R₄ and R₅ are double bonded, those of formula wherein R₁, R₂ and n are defined as above and either R₄ and R₅ are from a group consisting of hydrogen, hydroxyl (—OH) and acyloxyl of an organic carboxylic acid of up to 12 carbon atoms and R₆ and R₇ together form a double bond or are selected from a group consisting of hydrogen, hydroxyl (—OH) and acyloxyl of an organic carboxylic acid of up to 12 carbon atoms or R₅ and R₆ together to form a double bond and R₄ and R₇ are hydrogens and their non-toxic. More preferred compounds of formula 1 are those wherein R₁ and R₂ are both methyl, ethyl or isopropyl and those wherein n is 2 or 3 and their non-toxic, pharmaceutically acceptable acid salts. The compounds of this disclosure are not limited to the salt forms, but may include free base forms also.

The compounds of Structures I-IV have been shown to exhibit anti-breast cancer activities in assays involving MCF-7 cells. These compounds shown potent inhibition of the growth of MCF-7 breast cancer cells. These compounds can therefore be used in the treatment of breast cancer in warm-blooded animals. It is speculated that the compounds could be used to treat endometrial cancer as well.

The compounds can be administered in the manner suitable for medicinal or pharmacology purposes. The particular route of administration will depend on the kind of cancer and the dosage form chosen. The compounds of Structures I-IV can be administered orally, rectally, vaginally, parenterally or by local route such as sub-cutaneous in the breast of warm-blooded animals. Orally, all of the compounds can be in the form of tablets, capsules, granules, suppositories, injectable preparations and vaginal preparations.

### Pharmacological Tests

MCF-7 cells, purchased from ATCC (Rockville, Md.), were grown in RPMI medium 1640 with 10% FBS (GIBCO, Grand Island, N.Y.) and 1% Antibiotic-Antimycotic (GIBCO, Grand Island, N.Y.) in an incubator (NUAIRE NU-1500, Plymouth, Minn.) with 5% CO₂ and 95% air at 37 oC. Cells were grown as monolayer, doubling time is about 22 hours. The stock solution of the test compound was prepared at 1 mg/ml drug concentration. The stock solution was diluted serially to obtain the desired drug concentrations, e.g. 20, 100 and 200 μg/ml. Plates with twelve wells (5 ml capacity/well) were employed for all the experiments. In a typical experiment, each well contained 1×10⁵ initial cells and 100 μl drug solution in a total volume of 2 ml media. The final concentrations of compounds were 0, 10, 50 and 100 μg/ml in triplicate sample wells. The plates were incubated at 5% CO₂ and 37 oC for 72 hours under humid conditions. After incubation, cells were washed with PBS 1×, treated by 0.5 ml trypsin-EDTA for 10 minutes and the cells were suspended by adding 1.5 ml medium. Cells were counted by using a hemacytometer under a Nikon TMS 104 microscope. The data was converted to micromolar concentration using the various molecular weights and represent the mean values and standard errors.

Tables 1-4 illustrate the IC₅₀ (50% inhibitory concentration) values of compounds describe in this application against MCF-7 breast cancer cell line.

TABLE 1

| | IC₅₀ (micromolar scale) |
|---|---|
| Structure II Compounds | MCF-7 breast cancer cell line |
| (17β)-3-[2-dimethylamino]-ethoxy]- | 16.2 |
| Δ^{1,3,5(10)}-estrien-17-ol HCl salt | |
| (17β)-3-[2-diethylamino]-ethoxy]-]- | 8.7 |
| Δ^{1,3,5(10)}-estrien-17-ol HCl salt | |
| (17β)-3-[2-diisopropylamino]-ethoxy]-]- | 5.6 |
| Δ^{1,3,5(10)}-estrien-17-ol HCl salt | |
| (17β)-3-[2-morpholinyl]-ethoxy]-]- | 19.8 |
| Δ^{1,3,5(10)}-estrien-17-ol HCl salt | |
| (17β)-3-[2-piperidinyl]-ethoxy]-]- | 4.1 |
| Δ^{1,3,5(10)}-estrien-17-ol HCl salt | |
| (17β)-3-[2-pyrrolidinylamino]-ethoxy]-]- | 10.8 |
| Δ^{1,3,5(10)}-estrien-17-ol HCl salt | |
| 4-Hydroxytamoxifen HCl salt | 9.0 |

TABLE 2

| | IC₅₀ (micromolar scale) |
|---|---|
| | MCF-7 breast cancer |
| Structure III Compounds | cell line |
| 3-[2-dimethylamino]-ethoxy]-]- | 9.1 |
| Δ^{1,3,5(10)}-estrien-17α-ethynyl-17β-ol HCl salt | |
| 3-[2-diethylamino]-ethoxy]-]- | 7.3 |
| Δ^{1,3,5(10)}-estrien-17α-ethynyl-17β-ol HCl salt | |
| 3-[2-diisopropylamino]-ethoxy]-]- | 8.3 |
| Δ^{1,3,5(10)}-estrien-17α-ethynyl-17β-ol HCl salt | |
| 3-[2-morpholinyl]-ethoxy]-]- | 21.5 |
| Δ^{1,3,5(10)}-estrien-17α-ethynyl-17β-ol HCl salt | |
| 3-[2-piperidinyl]-ethoxy]-]- | 6.1 |
| Δ^{1,3,5(10)}-estrien-17α-ethynyl-17β-ol HCl salt | |
| 3-[2-pyrrolidinylamino]-ethoxy]-]- | 6.4 |
| Δ^{1,3,5(10)}-estrien-17α-ethynyl-17β-ol HCl salt | |
| 4-Hydroxytamoxifen HCl salt | 9.5 |

TABLE 3

| | IC₅₀ (micromolar scale) |
|---|---|
| Structure IV Compounds | MCF-7 breast cancer cell line |
| 3-[2-dimethylamino]-ethoxy]-]- | 16.2 |
| Δ^{1,3,5(10)}-estrien-17-one HCl salt | |
| 3-[2-diethylamino]-ethoxy]-]- | 8.7 |
| Δ^{1,3,5(10)}-estrien-17-one HCl salt | |
| 3-[2-diisopropylamino]-ethoxy]-]- | 5.6 |
| Δ^{1,3,5(10)}-estrien-17-one HCl salt | |
| 3-[2-morpholinyl]-ethoxy]-]- | 19.8 |
| Δ^{1,3,5(10)}-estrien-17-one HCl salt | |
| 3-[2-piperidinyl]-ethoxy]-]- | 4.1 |
| Δ^{1,3,5(10)}-estrien-17-one HCl salt | |
| 3-[2-pyrrolidinylamino]-ethoxy]-]- | 10.8 |
| Δ^{1,3,5(10)}-estrien-17-one HCl salt | |
| 4-Hydroxytamoxifen HCl salt | 9.0 |

The claimed compounds can be formulated into pharmaceutical compositions for use in the treatment of cancer cells. Standard pharmaceutical formulation techniques should be used as illustrated in acceptable pharmaceutical texts. A safe and effective amount of compounds relating to Stuctures I-IV should be administered. Safe and effective amount is an amount that effective enough to inhibit cancer cell growth in warm-blooded mammals, preferably in humans without severe side effects. The “safe and effective amount” of the compound effective to inhibit cancer cell growth may vary depending on the type of cancer cells, the stage of the disease, the physical and biological characteristics of the patient and the duration of the treatment.

The compositions of the subject invention contain at least one pharmaceutical carrier. The term “pharmaceutical carrier” means one or more compatible solid or liquid filler diluents or encapsulating substances which are suitable for administration to warm-blooded animals. The carriers should be sufficiently pure and have low toxicity to render them suitable for administration to warm-blooded animals. treated. Some examples of substances which can serve as pharmaceutical carriers are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid 5 and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutical carrier to be used in conjunction with the subject compound is basically determined by the way the compound is to be administered.

1. A method of treating cancer in warm-blooded animals by administering a safe and effective amount of a pharmaceutical composition comprising compounds selected from the group consisting of the compounds of Structure I 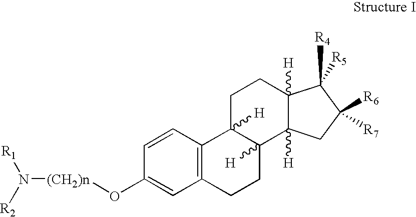
Wherein:
R₁ and R₂ are individually selected from the group consisting of alkyl groups containing 1 to 8 carbon atoms, taken together with nitrogen form a saturated 5 to 6 ring heterocycle,
R₃ is α or β methyl,
n is an integer from 2 to 10,
R₄ is selected from the group consisting of hydrogen, hydroxy and acyloxy of a organic carboxylic acid of up to 10 carbon atoms or taken together with the nitrogen form a saturated 5 to 6 ring heterocycle optionally containing a second nitrogen or oxygen in the ring, carboxylic acid and
R₅ selected from the group consisting of hydrogen, hydroxy, acyloxy of a organic carboxylic acid up to 10 carbons atoms and alkyl, alkenyl and alkynyl of up to 10 carbons,
R₆ and R₇ are individually selected from the group consisting of hydrogen, hydroxy and acyloxy of a organic carboxylic acid of up 10 carbons and
R₄ and R₇ are hydrogen and their non-toxic,
including pharmaceutically acceptable acid addition salts

2. The method of claim 1, wherein R₁ and R₂ are selected from the group consisting of methyl, ethyl, isopropyl, pyrrolidinyl cyclic ring, or morpholinyl cyclic ring or piperidinyl cyclic ring.

3. A method of treating cancer in warm-blooded animals by administering a safe and effective amount of a pharmaceutical composition comprising compounds selected from the group consisting of the compounds of Structure II 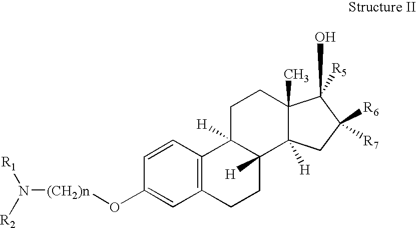
Wherein:
R₁ and R₂ are individually alkyl of 1 to 8 carbon atoms, taken together with nitrogen form a saturated 5 to 6 ring heterocycle,
n is an integer of 2 to 10 and
R₅ is hydrogen
R₆ is hydrogen
R₇ are hydrogen
including pharmaceutically acceptable acid salts.

4. The method of claim 3, wherein R₁ and R₂ are selected from the group consisting of methyl, ethyl, isopropyl, pyrrolidinyl cyclic ring, or morpholinyl cyclic ring or piperidinyl cyclic ring.

5. The method according to claim 4, wherein one or more of the compounds are selected from the group consisting of:
17β)-3-[2-dimethylamino]-ethoxy]-Δ^{1,3,5(10)}-estrien-17-ol
(17β)-3-[2-diethylamino]-ethoxy]-]-Δ^{1,3,5(10)}-estrien-17-ol
(17β)-3-[2-diisopropylamino]-ethoxy]-]-Δ^{1,3,5(10)}-estrien-17-ol
(17β)-3-[2-morpholinyl]-ethoxy]-]-Δ^{1,3,5(10)}-estrien-17-ol
(17β)-3-[2-piperidinyl]-ethoxy]-]-Δ^{1,3,5(10)}-estrien-17-ol
(17β)-3-[2-pyrrolidinylamino]-ethoxy]-]-Δ^{1,3,5(10)}-estrien-17-ol

6. A method of treating cancer in warm-blooded animals by administering a safe and effective amount of a pharmaceutical composition comprising compounds selected from the group consisting of the compounds of Structure III 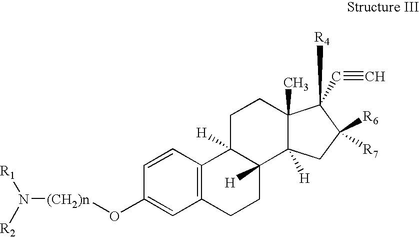
wherein:
R₁ and R₂ are individually selected from the group consisting of alkyl groups containing 1 to 8 carbon atoms, taken together with nitrogen form a saturated 5 to 6 ring heterocycle,
n is an integer of 2 to 10 and either R₄ is ethynyl,
R₆ is hydrogen,
R₇ are hydrogen
including their pharmaceutically acceptable acid salts.

7. The method of claim 6, wherein R₁ and R₂ are selected from the group consisting of methyl, ethyl, isopropyl, pyrrolidinyl cyclic ring, or morpholinyl cyclic ring or piperidinyl cyclic ring.

8. The method according to claim 6, wherein one or more of the compounds are selected from the group consisting of:
3-[2-dimethylamino]-ethoxy]-]-Δ^{1,3,5(10)}-estrien-17α-ethynyl-17β-ol
3-[2-diethylamino]-ethoxy]-]-Δ^{1,3,5(10)}-estrien-17α-ethynyl-17β-ol
3-[2-diisopropylamino]-ethoxy]-]-Δ^{1,3,5(10)}-estrien-17α-ethynyl-17β-ol
3-[2-morpholinyl]-ethoxy]-]-Δ^{1,3,5(10)}-estrien-17α-ethynyl-17β-ol
3-[2-piperidinyl]-ethoxy]-]-Δ^{1,3,5(10)}-estrien-17α-ethynyl-17β-ol
3-[2-pyrrolidinylamino]-ethoxy]-]-Δ^{1,3,5(10)}-estrien-17α-ethynyl-17β-ol

9. A method of treating cancer in warm-blooded animals by administering a safe and effective amount of a pharmaceutical composition comprising compounds selected from the group consisting of the compounds of Structure IV. 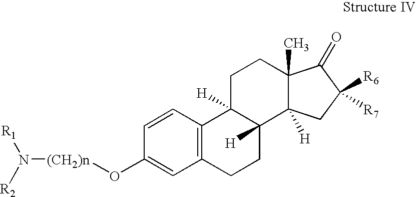
Wherein:
R₁ and R₂ are individually selected from the group consisting of alkyl groups containing 1 to 8 carbon atoms, taken together with nitrogen form a saturated 5 to 6 ring heterocycle,
n is an integer of 2 to 10,
R₄ and R₅ form a double-bond with oxygen (═O),
R₆ is hydrogen
R₇ is hydrogen
including their pharmaceutically acceptable acid salts.

10. The method of claim 9 wherein R₁ and R₂ are selected from the group consisting of methyl, ethyl, isopropyl, pyrrolidinyl cyclic ring, or morpholinyl cyclic ring or piperidinyl cyclic ring.

11. The method according to claim 9, wherein one or more of the compounds are selected from the group consisting of:
3-[2-dimethylamino]-ethoxy]-]-Δ^{1,3,5(10)}-estrien-17-one
3-[2-diethylamino]-ethoxy]-]-Δ^{1,3,5(10)}-estrien-17-one
3-[2-diisopropylamino]-ethoxy]-]-Δ^{1,3,5(10)}-estrien-17-one
3-[2-morpholinyl]-ethoxy]-]-Δ^{1,3,5(10)}-estrien-17-one
3-[2-piperidinyl]-ethoxy]-]-Δ^{1,3,5(10)}-estrien-17-one
3-[2-pyrrolidinylamino]-ethoxy]-]-Δ^{1,3,5(10)}-estrien-17-one

12. The method according to claim 1 wherein said cancer is breast cancer or endometrial cancer.

13. The method according to claim 3 wherein said cancer is breast cancer or endometrial cancer.

14. The method according to claim 6 wherein said cancer is breast cancer or endometrial cancer.

15. The method according to claim 9 wherein said cancer is breast cancer or endometrial cancer.

16. A pharmaceutical composition comprising:
(a) a safe and effective amount of a compound of Structure I according to claim 1 and
(b) a pharmaceutical carrier.

17. A pharmaceutical composition comprising:
(a) a safe and effective amount of a compound of Structure II according to claim 3 and
(b) a pharmaceutically-acceptable carrier.

18. A pharmaceutical composition comprising:
(a) a safe and effective amount of a compound of Structure III according to claim 6 and
(b) a pharmaceutical carrier.

19. A pharmaceutical composition comprising:
(a) a safe and effective amount of a compound of Structure IV according to claim 9 and
(b) a pharmaceutical carrier.
